(19) United States
(12) Patent Application Publication
(10) Pub. No.: US 2022/0105107 A1
(43) Pub. Date: Apr. 7, 2022

(54) BIOIDENTICAL PROGESTERONE CREAM INFUSED WITH NANOEMULSIFIED CBD

(71) Applicant: Michael Liguori, Port Orange, FL (US)

(72) Inventor: Michael Liguori, Port Orange, FL (US)

(21) Appl. No.: 17/060,551

(22) Filed: Oct. 1, 2020

(51) Int. Cl. A61K 31/57; A61K 31/05; A61K 9/16; A61K 9/06

(52) U.S. Cl. CPC A61K 31/57; A61K 9/06; A61K 9/16; A61K 31/05

(57) ABSTRACT

The present invention is a transdermal composition, for self-administering bioidentical hormone replacement and hormone balancing therapy and hormone balancing for both women and men, using a combination of progesterone and Cannabidiol (CBD) in various forms including nano-emulsified configurations. The compositions, which are topically applied by the user are trans dermally or topically delivered to the vascular system of the user in accordance with a dosage protocol established that will optimize the hormone levels of the user. Specific properties of CBD when combined with progesterone can provide an “entourage effect” that is produced from the synergistic interaction of the cannabinoids and progesterone in the user. The composition may also comprise various other excipients.

### CROSS REFERENCE TO RELATED APPLICATION

This US non-provisional utility patent application claims the benefit under 35 USC § 119(e) of U.S. provisional application No. 62/937,284 filed 19 Nov. 2020 which is expressly incorporated herein in its entirety by this reference.

### BACKGROUND OF THE INVENTION

### Field of the Invention

The present disclosure relates to hormone replacement therapy (HRT), bioidentical hormone replacement therapy and natural hormone balancing and, more particularly, to utilizing a compounded composition for affecting HRT, bioidentical hormone replacement therapy and natural hormone balancing with the complementary and innovative infusion of broad spectrum, full spectrum or isolate in various forms including nanosized (nano emulsified) configurations of cannabidiol (CBD), for treating the symptoms of peri menopausal, menopausal post menopausal and other symptoms of hormone imbalance in women as well as men, in general.

### Description of Related Art

Hormone Replacement Therapy (HRT), bioidentical hormone replacement therapy and natural hormone balancing are medical treatments to relieve symptoms of menopause. HRT, bioidentical hormone replacement therapy and natural hormone balancing involves the use of one or more of a group of medications designed to increase hormone levels in subjects who lack adequate hormone production. It can mitigate and prevent symptoms caused by diminished circulating estrogen and progesterone hormones in a pre-menopausal, peri-menopausal, menopausal, or post-menopausal subject.

Conventional hormone replacement therapy (HRT) is primarily administered through prescribed doses of synthetic drug preparations that mimic the action of the body's sex hormone molecules at the various receptor sites. The use of estrogens (Premarin) and synthetic progesterone (progestin) for hormone replacement therapy is common practice, but not without serious side effects that are well documented. Secondly, there are a handful of non-pharmaceutical grade creams containing bioidentical progesterone that are available in the marketplace but are not advancing or promoting a product with infused cannabidiol (CBD). These products typically are not tested in a lab and clinical environment and so results are typically subjective and anecdotal. Moreover, the use of cannabinoids in the field of HRT is uncommon and non-obvious.

The use of cannabinoids to treat medical illnesses is of great interest to the medical community. Cannabidiol, aka CBD, is one of 104 chemical compounds found in the cannabis plant. CBD can be derived from marijuana, or from its genetic cousin, hemp. Unlike THC (Tetrahydrocannabinol), CBD is not psychoactive, but has been proven to have many different therapeutic effects. CBD does so by interacting with the receptors of the human body's endocannabinoid system, which helps regulate pain, appetite, mood, memory, immunity, and sleep. Because CBD has been until very recently a schedule 1 substance banned by federal statute, scientific research is still in its early phases, but there is emerging evidence that CBD can help relieve chronic pain, anxiety and PTSD, Insomnia, symptoms associated with Multiple Sclerosis, epileptic seizures.

Attempts have been made at administering the cannabinoid Δ9-THC (Dronabinol) orally, in the form of a capsule. However, severely nauseated patients are often not able to retain the capsule in their stomachs long enough for the drug to take effect. Another issue with capsules, as well as Smoked marijuana, is that patients absorb the drug relatively rapidly (as compared to controlled drug delivery rates that a patch produces) and receive high drug concentrations in their body. These high drug concentrations, or peak levels, are often associated with serious psychoactive and other central nervous system side effects. There is also a need to trans dermally deliver cannabinoids other than Δ49-THC and Δ8-THC to treat various illnesses and/or symptoms.

### DESCRIPTION OF RELATED ARTS

Many therapeutics including progestin and/or cannabidiol and uses of such therapeutics in the treatment of endometriosis symptoms are well known. Examples of such therapeutics and methods are taught in US patent applications, U.S. Pat. No. 9,149,499B1 and U.S. Ser. No. 10/744,143B2 to Robinson. Further, it states cannabis based therapeutics, for use in treating those known to have or be at increased risk of endometriosis and other ailments. The disclosure in the above prior arts claims for a pharmaceutical pill or sub-dermally implantable pellet and the method thereof, for treating endometriosis.

An example of topical carrier containing progesterone in combination with one or more penetration enhancers have been disclosed in ‘Amadioet al., US patent application, US20190070197 A1’. ‘Rosensweet et al.’ claims a pharmaceutical two-phase bio-identical hormone admixture for hormone replacement therapy in the US patent application U.S. Ser. No. 10/660,905 B2 comprising of one or more bioidentical hormone formulations. In another art, ‘Stinchcomb et al.’ discloses pharmaceutical composition comprising cannabidiol and a penetration enhancer for treating and preventing diseases and disorders, as well as improving cosmetic appearance. ‘Bernick et al.’ in US patent application, U.S. Pat. No. 9,248,136B2 discloses transdermal hormone replacement therapy comprising solubilized progesterone alone and optionally with an estrogen for the treatment of pre, peri-menopausal, menopausal and post-menopausal females. Further ‘Bernicket et al’ discloses the natural combination hormone replacement formulations and therapies for treating hormonal imbalances.

Nevertheless, none of the prior arts explained above discloses the novel method of nano-emulsion/nano-technology by pairing a micronized progesterone drug with cannabidiol, CBD. The technical expertise employed in creating a “bioavailable” nanosized CBD compound in a laboratory testing environment combined with both bioidentical can escalate the medical and health benefits with different combinations and doses.

### SUMMARY OF INVENTION

The present invention discloses a Hormone balancing cream with the unique pairing of bio-identical progesterone and cannabidiol which is a novel method for treating the symptoms of menopause, peri-menopause and post-menopause and other symptoms of hormone imbalance.

According to an embodiment of the present invention, the present invention employs a new methodology for administering HRT by using a topical cream applied to the skin that includes in its list of ingredients, both USP Micronized Progesterone and CBD, known as Cannabis Sativa Extract by the International Nomenclature of Cosmetic Ingredients (INCI) and generally known in its various forms and strains including full spectrum, broad spectrum or an isolate all of which may be in both a nano emulsified or standard form. The method of use will be self-administered by users and will vary based on advice from their healthcare professional as well as individual ongoing experience. The bioidentical progesterone cream infused with CBD is delivered trans-dermally and/or topically to a user via topical administration.

According to yet another embodiment of the present invention, the present invention is directed to bioidentical hormone compositions formulated for administration and obviates one or more current limitations in the field by combining the main ingredient, bioidentical progesterone with CBD. This pairing is the result of a combination of the legal right to include CBD, based on the Hemp Farming Act of 2018, as well as insight, general knowledge and testing.

According to yet another aspect of the present invention, Nano emulsion or Nanotechnology is used as the methodology in the present invention, paired with a micronized progesterone drug and CBD. Nanotechnology manipulates atoms, molecules, and supramolecular formations, reducing their scale to between 1 and 100 nanometers (nm). This process reduces particles to one-millionth of their original size and even greater. The process of nanosizing increases the bioavailability of CBD and Progesterone exponentially and permits an easier distribution through the body and increases the delivery time.

According to an embodiment of the present invention, the neurochemical changes involving CBD will activate serotonin receptors and increases neuroprotective mechanisms and synaptic plasticity which will offset mood swings in the patient, and depression in the perimenopausal and post-menopausal users to maintain cognitive levels.

According to yet another embodiment of the present invention, the method for self-administering bioidentical hormone replacement therapy for both women and men using a combination of progesterone and CBD in various nano-emulsified configurations, sizes and dosages will produce desired results in various hormone balancing therapies

### BRIEF DESCRIPTION OF DRAWINGS

FIG. 1 illustrates “J” shaped curve according to an embodiment of the present invention.

FIG. 2 illustrates inverted “U” shaped curve according to an embodiment of the present invention.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are included to provide a thorough understanding of embodiments of the invention. One skilled in the relevant art will recognize, however, that the invention can be practiced without one or more of the specific details, or with other methods, components, materials, and so forth. In other instances, well-known structures, materials, or operations are not shown or described in detail to avoid obscuring aspects of the invention.

One embodiment of the invention is a topical cream or carrier comprising both bioidentical progesterone infused with CBD (Cannabidiol), for use in symptoms of menopause, perimenopausal and other symptoms of hormone imbalance, in a pharmacologically and/or neutraceutically acceptable vehicle capable for transdermal or topical delivery of the formulation.

In another embodiment of the present invention, the pharmaceutical composition provides an “entourage effect” that is produced from the synergistic interaction of the cannabinoids and progesterone in the system. The compositions, which are topically applied by the user are trans dermally or topically delivered to the vascular system of the user in accordance with a dosage protocol established that will optimize the hormone levels of the user.

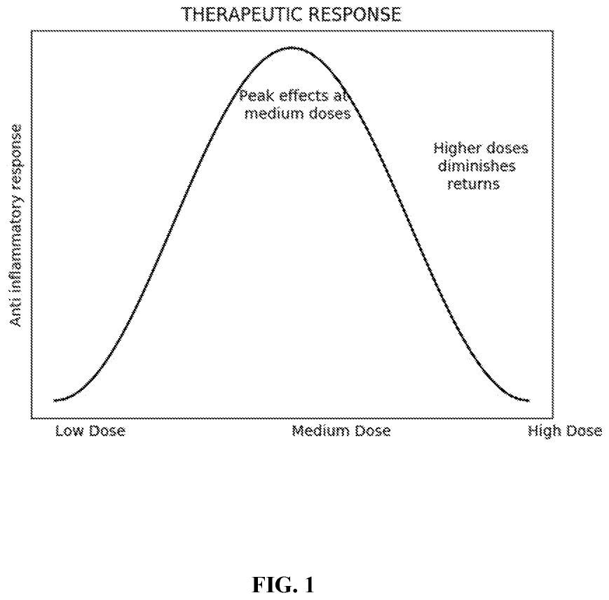

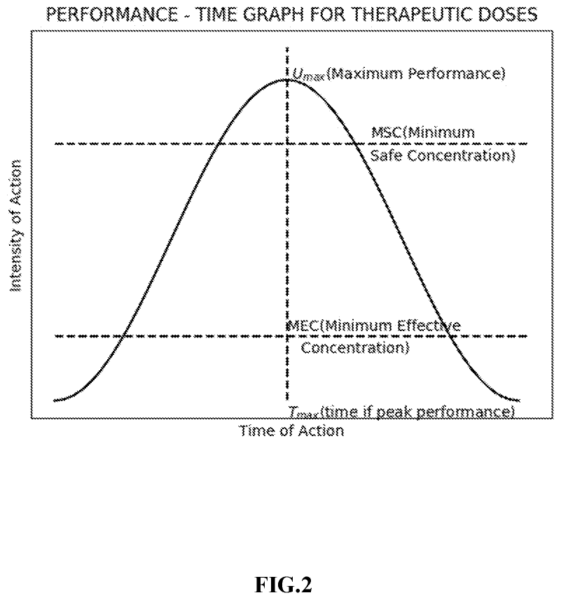

In yet another embodiment of the present invention, self-administering bioidentical hormone replacement therapy and hormone balancing with Cannabidiol (CBD) in its various forms including broad spectrum, full spectrum and isolate used for both genders. The idea behind choosing various forms of CBD is that when CBD is combined with a full-spectrum, broad-spectrum or an isolate (each will not have THC Tetrahydrocannabidiol), terps and phenolic, often see two results: lower doses are required, and the bell-shaped response curve as seen in FIG. 1 disappears (Although CBD is effective against inflammation and anxiety in rodent and human experiments, its effectiveness peaks at a medium dose but tapers off for higher doses and lower doses which graphically looks like an inverted “U” or a bell as shown in FIG. 2. Unfortunately, the bell-shaped curve means that CBD has limited usefulness). Full spectrum form of CBD provides firstly, a back-up support: Many terpenes and phenolic have effects similar to those of CBD on mood, pain and inflammation. Although CBD has more than 65 targets throughout the body, its activity at each individual target varies. Secondly, it provides complementary paths to the same outcome. When looking for relief from a complex issue like anxiety, there are often multiple paths that lead to the same outcome. While CBD is busy acting on serotonin receptors, other entourage compounds might focus on other neurotransmitters. Complementary pathways are effective against depression at lower doses when taken together in rodent experiments. Further, CBD provides counteracting downsides. Scientists think CBD has a bell-shaped dose response curve because it activates different targets at different concentrations. For instance, CBD relieves anxiety at moderate doses (through 5-HT1A receptors) but might trigger anxiety-inducing neurotransmitters at high doses (through TRPV1 receptors). In this way, the higher dose doesn't turn off the positive effects; it just turns on negative effects that obscure the benefits. However, the bell-shaped curve disappears when CBD is combined with molecules that block TRPV1 (which some natural flavonoids do). Further, full-spectrum CBD emits the property of bioavailability; CBD alone might not absorb or travel as far through the body without its entourage. A few of the terpenoids found in cannabis extracts are so good at moving drugs across skin barriers that they are added to products designed for transdermal delivery. In addition, the full-spectrum form of CBD enhances metabolism. Many members of the entourage are metabolized by the same enzymes as CBD (cytochrome p450). This means that the entourage could delay CBD from being broken down by the body keeping CBD in circulation and effective longer.

According to yet another embodiment of the present invention, the administration of a cream or other substance comprising bioidentical progesterone infused with CBD in a pharmacologically and/or neutraceutically acceptable vehicle operable for transdermal or topical delivery of the progesterone and CBD.

According to yet another embodiment of the present invention, the present invention employs the ‘nanoemulsion or nanotechnology’ methodology, which is being paired with a micronized progesterone drug and CBD. Nanotechnology manipulates atoms, molecules, and supramolecular formations, reducing their scale to between 1 and 100 nanometers (nm). This process reduces particles to one-millionth of their original size and even greater. It is another object of the present invention that nanosizing increases the bioavailability of CBD and progesterone exponentially and permits an easier distribution through the body and increases the delivery time.

According to yet another embodiment of the present invention, there is a sound technical expertise employed in creating a “bioavailable” nanosized CBD compound in a laboratory testing environment combined with both bioidentical progesterone and nanosized progesterone. It too can escalate the medical and health benefits with different combinations and doses and is safer to administer topically or trans dermally for treating hormone replacement therapy (HRT).

According to yet another aspect of the present invention, the present formulation treats perimenopausal and post-menopausal symptoms in woman synergistically. CBD is a potent anti-inflammatory that will decrease the levels of inflammatory molecules in the symptomatic subject. Osteoporosis has been linked to increased levels of TNF-alpha. CBD would lower the levels of this inflammatory molecule and reverse bone loss. CBD is also a potent vasodilator, increasing blood flow to the pelvic region, which in turn moisturizes the vagina canal; and as an anti-inflammatory relaxes the muscles and calming pain perceiving sensory nerves in the vulva and the vagina, with increased libido. The two ingredients progesterone and CBD synergistically work in tandem as they both produce these same characteristics.

It is an object of the present invention, wherein neuro-chemical changes involving CBD will activate serotonin receptors and increases neuroprotective mechanisms and synaptic plasticity which will offset mood swings in the patient, and depression in the perimenopausal and post-menopausal users to maintain cognitive levels. The following benefits, among others that may yet be discovered for Perimenopausal and Post-menopausal woman are synergistic and have qualities of uniqueness with respect to the pairing of CBD and progesterone: (a) Neurochemistry changes: As hormones decrease, they affect serotonin levels/receptors and other neurotransmitters and neuron generation in the hippocampus with fewer synaptic connections formed. CBD will activate serotonin receptors and increase neuroprotective mechanisms and increase synaptic plasticity which will offset mood swings, and depression in the perimenopausal and post-menopausal user and maintain cognitive levels. (b) As hormones decrease, levels of inflammatory molecules increase, specifically TNF-alpha (tumor necrosis factor) has been linked to cognitive problems and osteoporosis. Osteoporosis has been linked to a hormone deficiency and may return to normal with the pairing of progesterone and CBD.

Another object of the present invention is to cause CBD and progesterone to produce synergistic effect, as progesterone provides the same characteristics as other potent vasodilators and anti-inflammatory molecules, for treating the symptoms of perimenopausal and post-menopausal woman and other symptoms of hormonal imbalance. CBD is a potent vasodilator, increasing blood flow to the pelvic region, which in turn moisturizes the vaginal canal; and as an anti-inflammatory relaxes the muscles and calming pain perceiving sensory nerves in the vulva and the vagina, with increased libido.

It is further an object of the present invention to disclose the pharmaceutical composition as defined in any of the above, wherein the composition may also include various excipients such as Organic Aloe Barbadensis Leaf Juice, Organic Alcohol, Organic Carnauba Wax, Organic Glycerin, Organic Butyrospermum Parkii (Shea) Butter, Organic Cocos Nucifera (Coconut) Oil, Organic Helianthus Annuus (Sunflower) Seed Oil, Organic Prunus Amygdalus Dulcis (Sweet Almond) Oil, Organic Theobroma Cacao (Cocoa) Seed Butter, Sambucus Nigra Fruit Extract, Xanthan Gum and Sodium Citrate.

It is a further object of the present invention to disclose the pharmaceutical composition as defined in any of the above detailed manner, wherein the composition is formulated in a sustained release dosage form or in a rapid release dosage form or in a combination thereof.

### DEFINITIONS USED

### Bioidentical Progesterone

Bioidentical progesterone, which is used in the description above, is simply progesterone. The interest in a more natural approach to hormone therapy has focused attention on bioidentical hormones—hormones that are identical in molecular structure to the hormones women make in their bodies. Each are not found in this form in nature but are made, or synthesized, from a plant chemical extracted from yams and soy. Bioidentical progesterone, which is used in the description above, is simply progesterone. It is micronized (finely ground) in the laboratory for better absorption in the body. The term “micronized progesterone,” as used herein, includes micronized progesterone having an X50 particle size value below about 15 microns and/or having an X90 particle size value below about 25 microns.

Reference to progesterone as used herein, both naturally-occurring and bio-identical, is generally collectively referred to herein as “progesterone.” Various references generally suggest the use of the progestogen class of hormones for inclusion in transdermal therapies, typically in combination with an estrogen. The teachings within such references are generally limited to progestin however. Progestins are sometimes confused in the literature with progesterone or progestogen generally. Yet progestins are a distinct subclass of progestogen; a synthetic progestogen that has progestational effects similar to progesterone. Typically, synthetic progestins have higher activity and are administered at significantly lower dosages relative to natural progesterone.

The term “progestogen(s),” as used herein, is inclusive of naturally-occurring and bio-identical progesterone and all synthetic progestins. The term “bio-identical” hormones, as used herein, refers to hormones which are identical in chemical structure to the hormones naturally produced by mammals, including humans, and can be used, and are often referred to as, natural hormone replacement therapy, or “NHRT.”

### Nano-Emulsion

Nano-emulsion or nanotechnology as used herein is the technology employed in the pharmaceutical formulation. Self-micro- or self-nano-emulsifying drug delivery systems can produce nanoemulsion (NE) and improve the oral bioavailability of highly lipophilic compounds. CBD is highly lipophilic, and it is usually supplied as an oil preparation.

The term “oil,” as used herein may be any pharmaceutically acceptable substance, other than peanut oil, that would suspend and/or solubilize any suitable progestogen, including progesterone, starting material, or precursor, including micronized progesterone as described herein. More specifically, oils may include, for example and without limitation, medium chain fatty acids, generally of the group known as medium chain fatty acids consisting of at least one mono-, di-, and triglyceride, or derivatives thereof, or combinations thereof.

### Nutraceutical

“Nutraceutical” is a substance that may be considered a food or part of a food which provides medical or health benefits, encompassing prevention and treatment of disease. Products as diverse as isolated nutrients, dietary supplements and diets to genetically engineered “designer” foods, herbal products, and processed foods (cereals, soups, beverages) may be included under the umbrella of nutraceuticals. Nutraceuticals are a group of products that are more than food but less than pharmaceuticals.

### Entourage Effect

The entourage effect as used herein, is a proposed mechanism by which cannabis compounds other than Tetrahydrocannabinol (THC) act synergistically with it to modulate the overall psychoactive effects of the plant. In the context of the invention, it refers to producing synergistic interaction of the cannabinoids and progesterone in the vascular system of the patient, especially; it acts on symptoms of menopause such as pelvic pain and other symptoms of hormonal imbalances.

The word/words “Formulation” and Composition” have been used interchangeably in the present specification.

In view of the above description, the formulation of bioidentical progesterone infused with cannabinoids is be delivered trans dermally or topically (across the skin). Preferably, such formulation is delivered to the vascular system of the patient in accordance with a dosage protocol established that will optimize the hormone levels of the user.

What is claimed is:

1. A transdermal pharmaceutical composition for self-administration of a bioidentical hormone replacement and natural hormone balancing therapy in both genders essentially comprising of:
a) bioidentical progesterone hormone;
b) micronized progesterone hormone;
c) nanosized progesterone hormone;
d) nanosized cannabidiol (CBD);
e) both broad spectrum full spectrum, and isolate forms of cannabidiol (CBD), wherein the said composition is capable of providing an entourage effect into the vascular system of the human body.

2. The transdermal pharmaceutical composition of claim 1, wherein the said pharmaceutical composition uses nano-emulsion technology for effective absorption of the said transdermal pharmaceutical composition into the vascular system of the human body.

3. The transdermal pharmaceutical composition of claim 1, wherein the said forms of cannabidiol enhances bioavailability in the human body.

4. The transdermal pharmaceutical composition of claim 1, wherein the said form of CBD includes a non-psychoactive component that interacts with the endocannabinoid receptors of the human body.

5. The transdermal pharmaceutical composition of claim 1, wherein the said bioidentical progesterone hormone is micronized progesterone having particle size value below 15 microns.

6. The transdermal pharmaceutical composition of claim 1, wherein self-administration of the composition means transdermal or topical application of the progesterone and nano-emulsified cannabidiol (CBD).

7. The transdermal pharmaceutical composition of claim 1, wherein the said bioidentical progesterone hormone infused with CBD produces a synergistic effect for treating peri-menopausal and postmenopausal symptoms in women.

8. The transdermal pharmaceutical composition of claim 1, wherein CBD is a potent vasodilator, increases blood flow in pelvic region, moisturizes vaginal canal, and relaxes anti-inflammatory muscles, calming sensory nerves in vulva of women.

9. The transdermal pharmaceutical composition of claim 1, wherein CBD activates serotonin receptors which help in to maintaining cognitive levels in the brain.

10. The transdermal pharmaceutical composition of, wherein the said ingredients may include any of these or combination of Organic Aloe Barbadensis Leaf Juice, Organic Alcohol, Organic Carnauba Wax, Organic Glycerin, Organic Butyrospermum Parkii (Shea) Butter, Organic Cocos Nucifera (Coconut) Oil, Organic Helianthus Annuus (Sunflower) Seed Oil, Organic Prunus Amygdalus Dulcis (Sweet Almond) Oil, Organic Theobroma Cacao (Cocoa) Seed Butter, Sambucus Nigra Fruit Extract, USP Micronized Progesterone, Cannabis Sativa Extract (Nano-Emulsified CBD), Xanthan Gum, Sodium Citrate.

11. A method of formulating a transdermal cream for self-administration of a bioidentical hormone replacement and natural hormone balancing therapy for the medical treatment in both genders essentially comprising of:
a) bioidentical progesterone hormone;
b) micronized progesterone hormone;
c) nanosized progesterone hormone;
d) nanosized cannabidiol (CBD);
e) both broad spectrum, full spectrum and isolate forms of cannabidiol (CBD), wherein the said composition is capable of providing an entourage effect into the vascular system of the human body.

12. The method for formulating a transdermal cream according to claim 11, wherein the said cream is administered for topical or transdermal delivery into the vascular system of the human body.

13. The method of formulating the transdermal cream of claim 11, wherein the said method essentially produces a synergistic effect for treating peri-menopausal and postmenopausal symptoms.

14. The method of formulating the transdermal cream of claim 11, wherein the said nano-emulsion technology is achieved by pairing a bioidentical micronized progesterone hormone with cannabidiol (CBD).

15. The method of nano-emulsion technology of claim 11, wherein the said nano-emulsion technology refers to nanosizing of particles increasing the bioavailability of CBD and [progesterone exponentially by permitting easier distribution through the vascular system enhancing the delivery time.

16. A transdermal pharmaceutical composition according to claim 1 for treating patients suffering from a condition selected from a group comprising of menopausal, peri-menopausal and postmenopausal symptoms and other hormonal imbalances symptoms such as osteoporosis, insomnia, chronic pain, anxiety, PTSD, inflammation, depression, symptoms associated with Multiple Sclerosis, and epileptic seizures.
